(19) United States
(12) Patent Application Publication
(10) Pub. No.: US 2025/0381070 A1
(43) Pub. Date: Dec. 18, 2025

(54) GOGGLE STRUCTURE

(71) Applicant: ARGUS VISION INTERNATIONAL LIMITED, Tainan (TW)

(72) Inventor: Lin -Yun Chen, Tainan (TW)

(21) Appl. No.: 19/218,384

(22) Filed: May 26, 2025

(30) Foreign Application Priority Data
Jun. 17, 2024 (TW) 113122368

(51) Int. Cl. A61F 9/02; A42B 3/18

(52) U.S. Cl. CPC A61F 9/025; A42B 3/185

(57) ABSTRACT

Provided is a goggle structure, which includes a lens, a frame, two adjustable linkage sets and two wearing bases. A frame is provided with a pivot base on each side. Each of the adjustable linkage sets includes a first arm body and a second arm body pivoted at one end to each other. Another end of the first arm body is pivoted on the pivot base. The second arm body is fixed to a side of the lens. The two wearing bases are respectively provided on both sides of the frame. The lens is operable to form spacing from the frame by means of the pivoting of the first arm body and the second arm body.

### BACKGROUND OF THE INVENTION

### 1. Field of the Invention

The present disclosure relates to a type of goggle, and particularly relates to a structure in which a distance between a lens and a frame can be adjusted and the lens can be operatively lifted up.

### 2. The Prior Arts

In general, when doing extreme sports such as skateboarding, cycling, surfing, skiing, etc., most of them maintain a certain speed, and oncoming wind or snow will seriously affect your vision. Walking while not being able to see clearly in front of you increases the risk of injury from falling or hitting others, and reduces the enjoyment of the exercise. Therefore, goggles play an important role in ensuring good vision.

Referring to FIG. 1, the structure of the existing goggles 5 mainly consists of a lens 51 fixed to the front side of a frame 52. An elastic band 53 is attached to both sides of the frame 52. The wearer attaches the frame 52 to the face, and ties the elastic band 53 around the head. Alternatively, the wearer wearing a helmet C secures the elastic band 53 to the outside of the helmet C, thereby protecting the eyes from being affected during exercise/movement.

However, when the speed of heat generated by exercise/movement is not equal to the speed of heat carried away by airflow, the lens 51 is prone to fogging. When the fogging affects the wearer's vision, the wearer can only slow down the movement speed or move the goggles 5 to the forehead for a short period of time by holding the frame 52. Alternatively, as shown in FIG. 1, the goggles 5 are moved to the front side of the helmet C (as shown in the chain line), and the goggles 5 will not be detached from the head or the helmet C when moved by the elastic binding of the elastic band 53, and the goggles 5 will be restored to the previous position after the fog has cleared. Further, when taking a break in the middle of exercise or when the lens 51 is not needed to protect the eyes, the wearer has to move the goggles 5 upward to the forehead or to the front side of the helmet C. This is quite inconvenient for the wearer r and may affect the enjoyment of the exercise.

### SUMMARY OF THE INVENTION

In view of the shortcomings and deficiencies of the prior art, the present disclosure provides a structure for increasing air flow by adjusting a distance between a lens and a frame, and a structure that can be operated to lift the lens upward.

In order to achieve the above-mentioned objective, the technical means adopted in the present disclosure is a goggle structure, which mainly includes: a lens, a frame, two adjustable linkage sets and two wearing bases.

In an embodiment, the lens is provided.

In an embodiment, the frame is provided with a pivot base on each side.

In an embodiment, two adjustable linkage sets are symmetrically pivoted on each side of the frame. Each of the adjustable linkage sets comprises a first arm body and a second arm body pivoted at one end to each other. Another end of the first arm body is pivoted on the pivot base and is operable to pivot relative to the pivot base, and the second arm body is fixed to a side of the lens and is operable to pivot relative to the first arm body

In an embodiment, two wearing bases are symmetrically respectively provided on both sides of the frame for a wearing element to be assembled.

In an embodiment, the lens is operable to form spacing from the frame by means of the pivoting of the first arm body and the second arm body.

### BRIEF DESCRIPTION OF THE DRAWINGS

FIG. 1 is a schematic diagram showing the prior art.

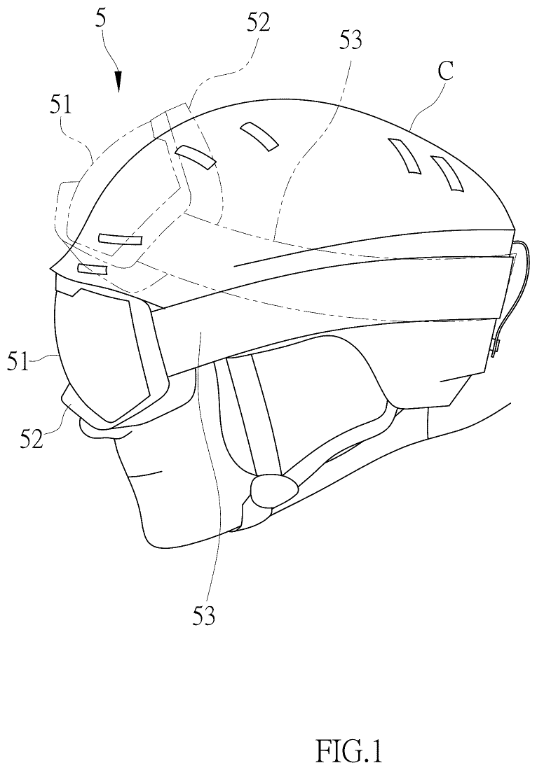

FIG. 2 is a schematic diagram showing the decomposition of a goggle structure according to the present disclosure.

FIG. 3 is a schematic diagram showing the partial decomposition of a frame and a first arm body according to the present disclosure.

FIG. 4 is a schematic diagram showing the partial decomposition of a lens and a second arm body according to the present disclosure.

FIG. 5 is a schematic diagram showing the partial decomposition of the frame and the lens according to the present disclosure.

FIG. 6 is a schematic diagram showing the partial decomposition of a wearing base and the frame according to the present disclosure.

FIG. 7 is a schematic diagram showing the three-dimensional assembly of the goggle structure according to the present disclosure.

FIG. 8 is a schematic cross-sectional view of the wearing base and the frame according to the present disclosure.

FIG. 9 is a schematic longitudinal cross-sectional view of the goggle structure according to the present disclosure.

FIG. 10 is a schematic diagram showing the distance/spacing between the lens and the frame according to the present disclosure.

FIG. 11 is a schematic diagram showing the lens lifted upward according to the present disclosure.

FIG. 12 is a schematic partially enlarged diagram of FIG. 9.

### DETAILED DESCRIPTION OF THE PREFERRED EMBODIMENT

The following is a specific embodiment to illustrate the implementation of the present disclosure. Persons skilled in the art can easily understand the other advantages and effects of the present disclosure from the disclosure in the specification.

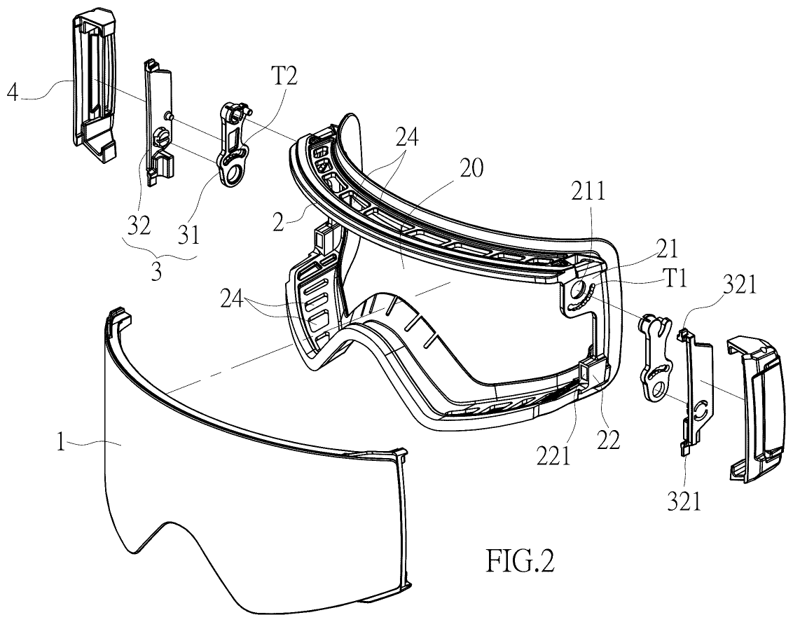

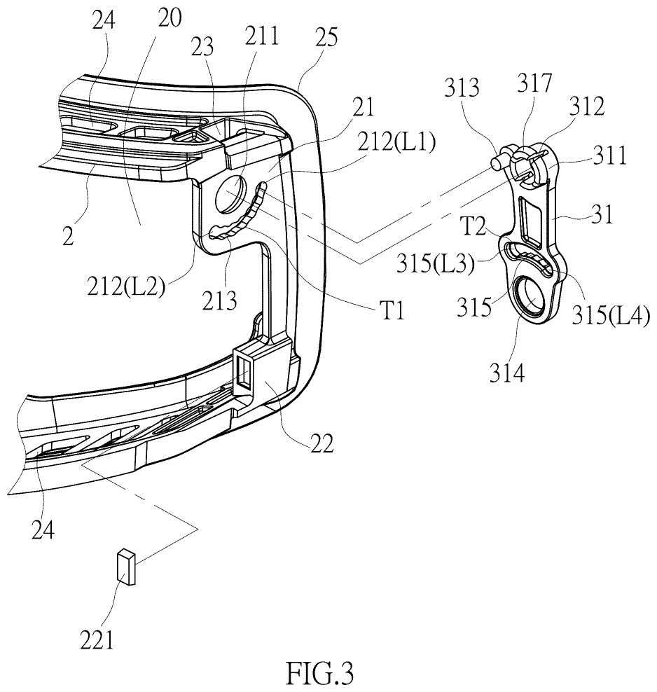

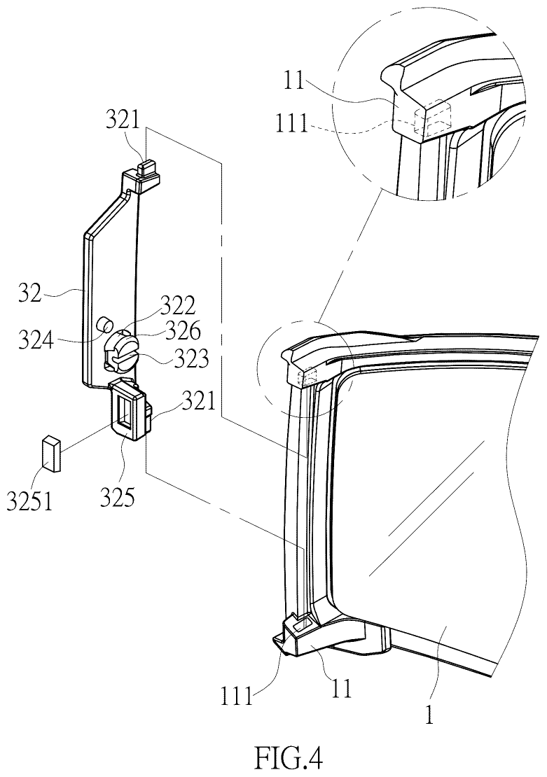

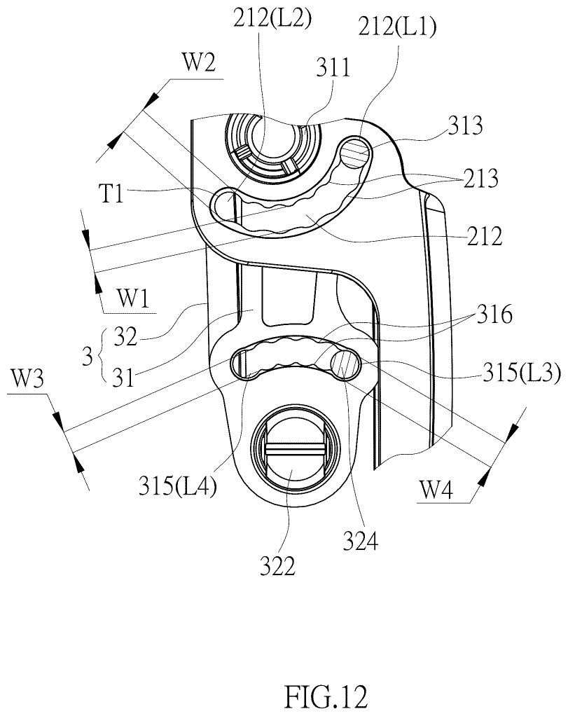

Referring to FIG. 2 to FIG. 7 and FIG. 12, in a preferred embodiment, the present disclosure provides a goggle structure, in which a side facing a wearer's face is defined as an inner side, and the side not facing the wearer's face is defined as an outer surface. The goggle structure mainly includes a lens 1, a frame 2, two adjustable linkage sets 3 and two wearing bases 4.

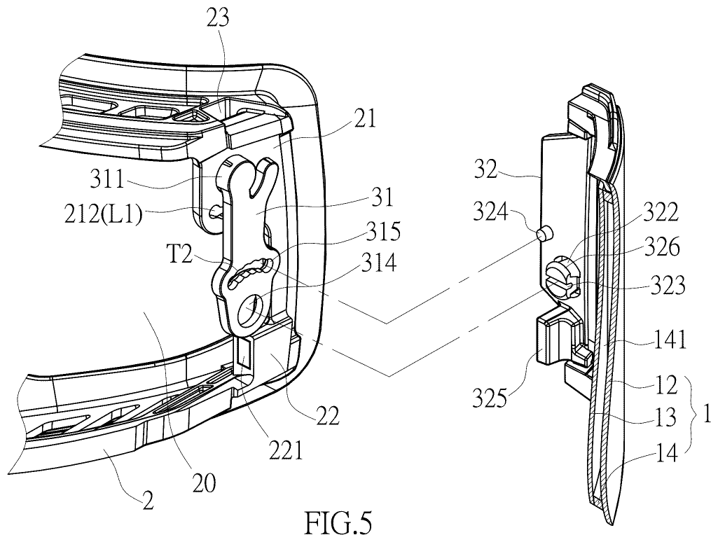

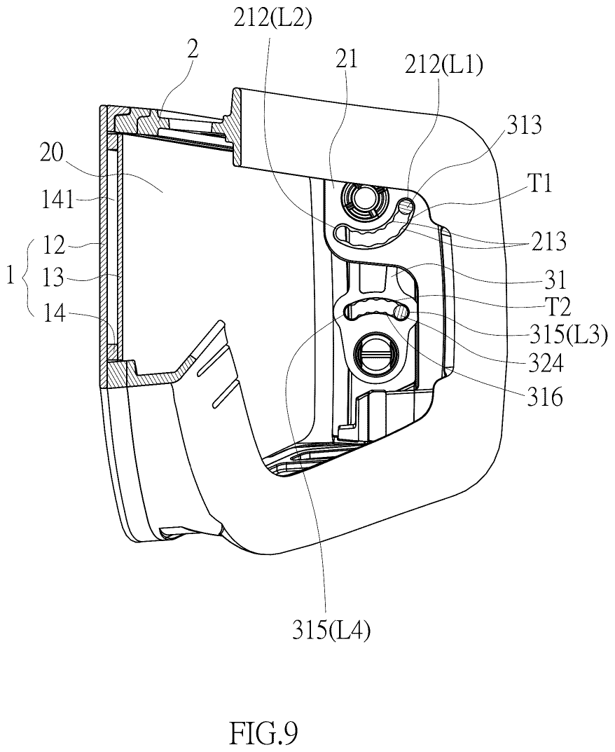

The upper and lower edges of the two sides of the lens 1 are provided with opposing hole bases 11, each of which is provided with a fixing hole 111. Each fixing hole 111 is polygonal in shape. The drawings in the present disclosure are illustrated in the form of a quadrilateral as an example, but not limited thereto. Each of hole bases 111 is provided on both sides of the lens 1 by one of the following methods: adhesive, fusion, locking, embedding, integral extension, or secondary injection. Moreover, the lens 1 may be a single-layer or a double-layer lens, preferably a double layer lens as shown in FIGS. 5 and 9, which show that the lens 1 has a front lens 12 and a rear lens 13 side by side, a spacer pad 14 is provided between the front lens 12 and the back lens 13, and the spacer pad 14 is disposed circumferentially around the periphery of the front lens 12 and back lens 13, thereby allowing the front lens 12 and the rear lens 13 to form a hollow temperature insulation layer 141 without affecting the line of sight, effectively blocking the temperature difference between the outside and inside of the lens 1, and reducing the fogging problem of the lens 1. The lens 1 may be a flat lens, a prescription lens, a sunshade lens, an eye protection lens, or one of the color-changing lenses. In addition, when the lens 1 is a single-layer lens, it may be a one-sided lens or a left-right symmetrical two-sided lens.

The frame 2 encloses a hollow field of view 20, and a pivot base 21 is provided on each side of the frame 2. In an embodiment, the pivot base 21 is provided at the upper edge of both sides of the frame 2, and extends integrally from the frame 2. The pivot base 21 has a first pivot hole 211. The pivot base 21 is provided with a plurality of connected first adjusting holes 212 adjacent to the first pivot hole 211. A first neck 213 is formed between two adjacent first adjusting holes 212. The first adjusting holes 212 collectively defines a first arcuate track T1, and two ends of the first arcuate track T1 are defined as a first position L1 and a second position L2 respectively. In an embodiment, the first neck 213 is an upward and downward curved shape, such that the width W1 of the first neck 213 is smaller than the width W2 of each of the first adjusting holes 212. Also, the lower edge of each side of the frame 2 is provided with a first magnet base 22. The first magnet base 22 is provided with a first magnet 221, and the first magnet 221 is oriented toward the outer side of the frame 2. The top and bottom of both sides of the frame 2 are provided with snap holes 23, and the frame 2 is provided with a plurality of spaced apart ventilation holes 24 between the snap holes 23 at the top and between the snap holes 23 at the bottom. In addition, the inner side of the frame 2 has a cushion 25, which may be made of plastic or flexible material, cushioning material, such as rubber, silicone, foam, etc.

The adjustable linkage set 3 is symmetrically pivoted on each side of the frame 2, and each adjustable linkage sets 3 includes a first arm body 31 and a second arm body 32. A first hook 311 is provided on the inner side of the upper end of the first arm body 31. In an embodiment, the first hook 311 is in the shape of a cylinder. The end portion of the first hook 311 is provided with a first protrusion 317 annularly outwardly, and the end portion of the first hook 311 is provided with a plurality of first slots 312 spaced axially. The first slots 312 serve as space for elastic compression of the first hook 311. The first hook 311 is forced into the first pivot hole 211 with the end portion thereof and is hooked to the inner side of the pivot base 21 by the first protrusion 317, such that the first arm body 31 is secured to each side of the frame 2 and can pivot relative to the pivot base 21.

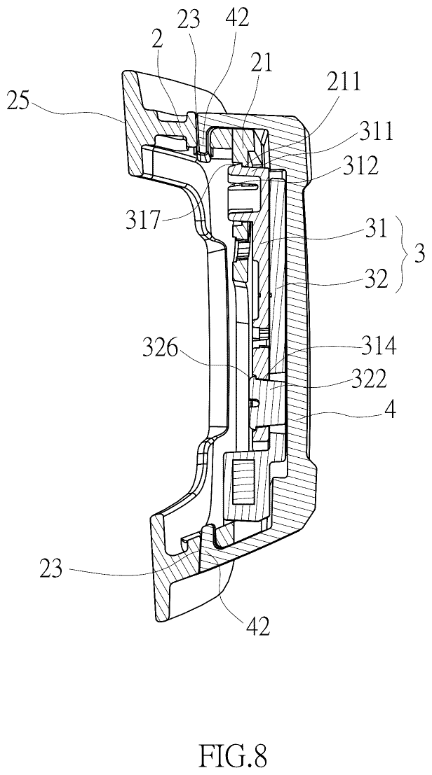

The first arm body 31 is provided with a first positioning portion 313 extending integrally from the inner side of the upper end of the first arm body 31. In an embodiment, the first positioning portion 313 may be a column, but not limited thereto. The first positioning portion 313 is selectively embedded in one of the first adjusting holes 212. FIG. 2 to FIG. 9 show that the first positioning portion 313 is embedded in the first position L1 of the first adjusting holes 212. In addition, the lower end of the first arm body 31 is provided with a second pivot hole 314. The first arm body 31 is provided with a plurality of connected second adjusting holes 315 adjacent to the second pivot hole 314. A second neck 316 is formed between two adjacent second adjusting holes 315. The second adjusting holes 315 collectively defines a second arcuate track T2, and two ends the second arcuate track T2 are defined as a third position L3 and a fourth position L4, respectively. In an embodiment, the second neck 316 is an upward and downward curve shape, such that the width W3 of the second neck 316 is smaller than the width W4 of each of the second adjusting holes 315.

The second arm body 32 is secured to each side of the lens 1, and the present disclosure does not limit the manner in which the second arm 32 is fixed to each side of the lens 1. In an embodiment, the upper and lower edges of the second arm body 32 are each integrally extended with an inserting member 321. The inserting member 321 is inserted into the corresponding fixing hole 111 to form a fixing, and the profile of the inserting member 321 matches the shape of the fixing hole 111 of the lens 1. The inner side of the second arm body 32 is provided with a second hook 322. In an embodiment, the second hook is in the shape of a cylinder. The end portion of the second hook 322 is provided with a second protrusion 326 annularly outwardly, and the end portion of the second hook 322 is axially provided with a second slot 323. The second slot 323 serves as an elastic compression space for the second hook 322. The second hook 322 is operable to pivot relative to the first arm body 31 by forcing the end portion thereof into the second pivot hole 314 and by hooking the second protrusion 326 to the inner side of each of the first arm body 31. The second arm body 32 has a second positioning portion 324. In an embodiment, the second positioning portion 324 may be a column. The second positioning portion 324 is selectively embedded in one of the second adjusting holes 315. FIG. 5 to FIG. 9 show that the second positioning portion 324 is embedded in the second adjusting hole 315 at the third position L3. Also, the second arm body 32 is provided with a second magnet base 325, which is provided with a second magnet 3251. The second magnet 3251 is magnetically attracted to or separated from the first magnet 221 with the operation of the lens 1.

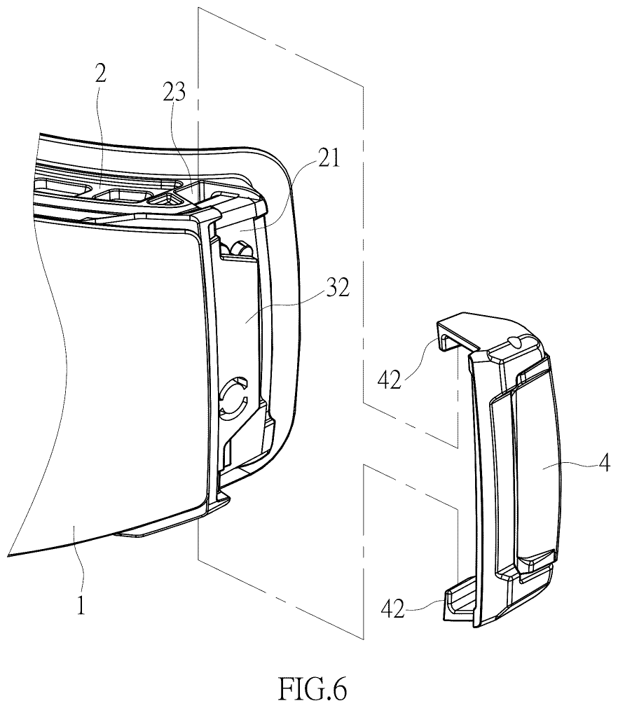

Two wearing bases 4 are symmetrically respectively disposed on both sides of the frame 2, and can be assembled with a wearing element 41, such as a strap. In an embodiment, snap hooks 42 extend from the top and the bottom of each of the wearing bases 4 inwardly, as shown in FIG. 6 to FIG. 8. Each of the wearing bases 4 covers the outer side of each of the adjustable linkage sets 3, and is secured by each of the snap hooks 42 by means of hooking from to the outer side of the frame 2 into the corresponding snap holes 23 thereof.

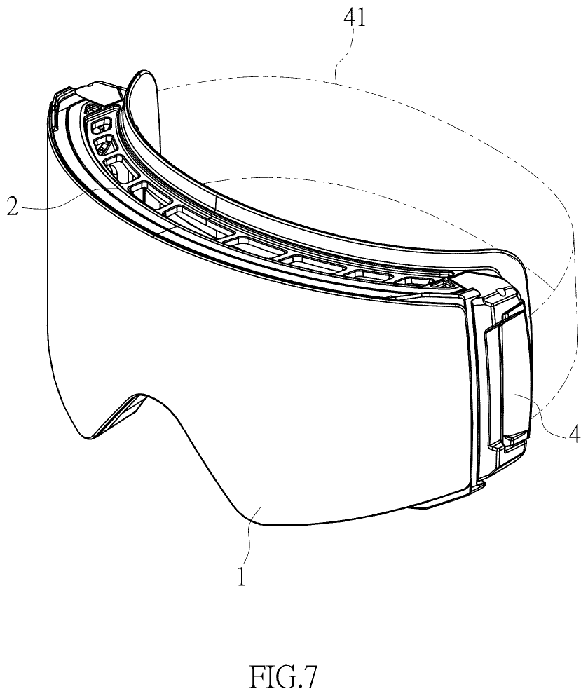

Hereinafter, in a normal state of the present disclosure, as shown in FIG. 7 and FIG. 9, each of the first positioning portions 313 is embedded in the first adjusting hole 212 at the first position L1, and each of the second positioning portions 324 is embedded in the second adjusting hole 315 at the third position L3, such that each of the second magnets 3251 is magnetically attracted to each of the first magnet 221, and the lens 1 is adhered to the outside of the frame 2.

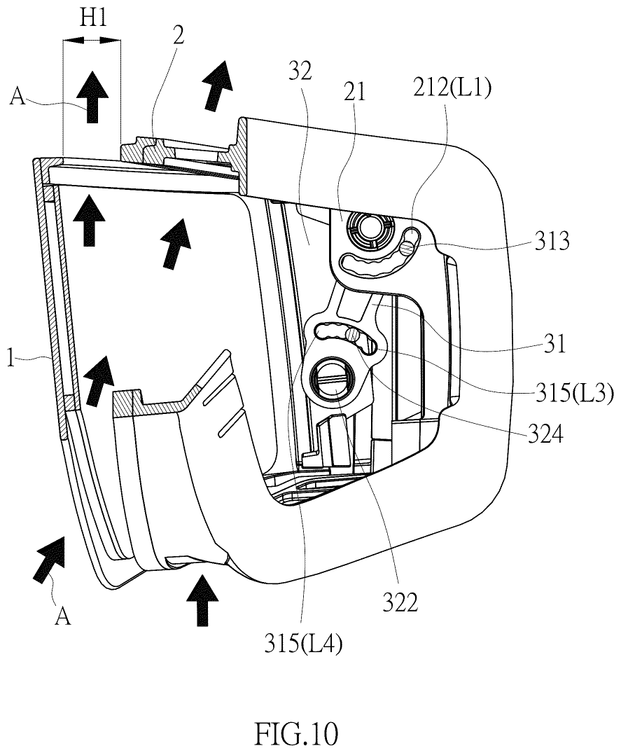

Continuing with reference to FIG. 9 and FIG. 10, when the speed of the airflow from the ventilation holes 24 to take away the hot air inside the frame 2 is too slow, the wearer only needs to hold the lens 1 and exert a force outwardly, such that the second arm body 32 pivots relative to the first arm body 31, and the first arm body 31 pivots relative to the frame 2, so as to a distance/spacing H1 between the lens 1 and the frame 2, thereby increasing the path of air A into the frame 2, enhancing the speed of convection, which can quickly taking away the hot air inside the frame 2, and reducing the fogging problem of the lens 1. When the wearer applies an outward force on the lens 1, the angle range of pivoting of each of the adjustable linkage sets 3 is known by the feeling of resistance operation of the second positioning portion 324 that passes through the narrower second neck 316 and is embedded in the adjacent second adjusting holes 315, and the feeling of resistance operation of the first positioning portion 313 that passes through the narrower first neck 213 and is embedded in the adjacent first adjusting holes 212, thereby adjusting the distance/spacing Hl between the lens 1 and the outer side of the frame 2 by hand.

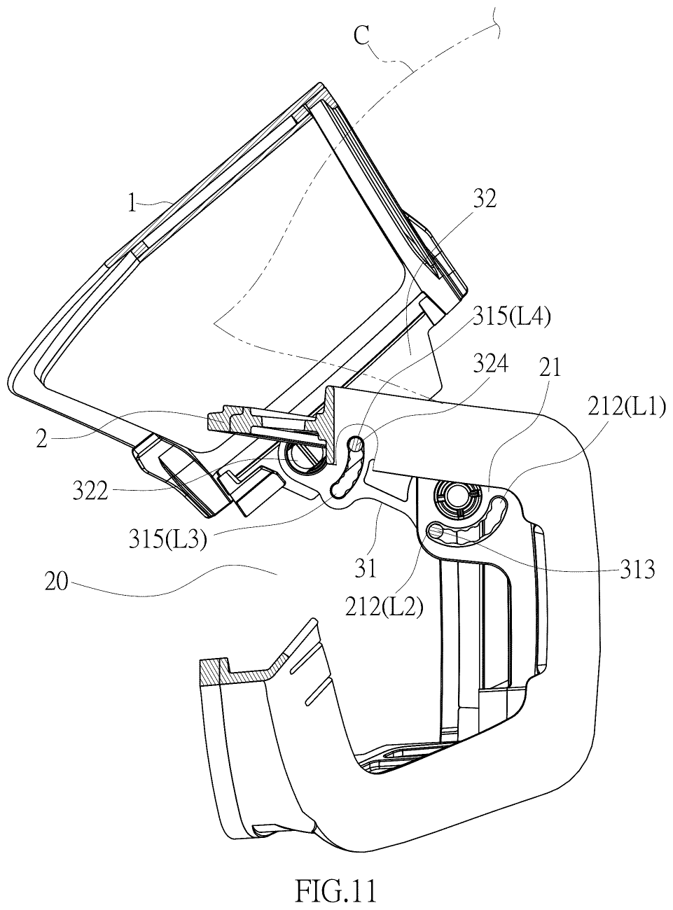

As described above and with reference to FIG. 9 to FIG. 11, when taking a break in the middle of exercise, or when the wearer does not need the lens 1 to protect the eyes for the time being, the wearer only needs to hold the lens 1 and apply force outwardly and upwardly, such that the first arm body 31 pivots with respect to the frame 2, and such that the first positioning portion 313 moves in the direction of the first position L1 along the first arcuate track T1. The second arm body 32 is pivoted relative to the first arm body (i.e., moved in the direction from the first position L1 to the second position L2 along the first adjusting holes 212), such that the second positioning portion 324 is moved in the direction from the third position L3 to the fourth position L4 along the second arcuate track T2. By means of the above-described feeling of resistance, the angle at which the lens 1 is lifted up towards the upper edge of the frame 2. FIG. 11 is a schematic diagram showing the movement of the first positioning portion 313 from the first position LI to the second position L2 and the movement of the second positioning portion 324 from the third position L3 to the fourth position L4. However, instead of restricting the upward lifting angle of the lens 1, the wearer may selectively operate the first positioning position 313 embedded in one of the first adjusting holes 212 to form a fixation and operate the second positioning portion 324 embedded in one of the second adjusting holes 315 to form a fixation according to the comfort of the wearer's visual perception. The lens 1 is then lifted up at an angle, such that the hollow field of view of the frame 2 is not obscured by the lens 1. That is to say, the present disclosure does not need to move the whole frame including the lens to the forehead or the front side of the helmet C as in the conventional technology.

It is worth mentioning that the present disclosure allows the lens 1 to be operatively pivoted outwardly by the continuous pivoting of the first arm body 31 and the second arm body 32, such that the lens is directly lifted up on the front side of the helmet C without interfering with the hood of the helmet C, thereby improving the convenience of use.

Furthermore, the second arm body 32 is elastically forced into the second pivot hole 314 of the first arm body 31 by the second hook 322. Therefore, when the wearer wants to replace the lens 1, the second hook 322 can be operatively forced out of the second pivot hole 314, and the lens 1 can be detached from the frame 2 for replacement.

In summary, the present disclosure has the following advantages over the prior art.
1. The distance between the lens and the frame can be adjusted without the aid of tools, thereby increasing air convection and reducing fogging of the lens.
2. The adjustable linkage set has a linkage pivot function, such that the lens can be operated relative to the frame upward lifting, thereby exposing the field of view of the frame. In addition, the upward lifting process will not interfere with the helmet.
3. The lens can be removed from the frame for replacement without the aid of tools.

As such, according to the aforementioned structure, the present disclosure solves the inconvenience of the conventional technology and allows the wearer to lift the lens up by simple operation when doing extreme sports such as skateboarding, cycling, surfing, skiing, etc. The present disclosure can also increase the air flow and reduce the generation of fog.

Although the present disclosure has been described with reference to the preferred exemplary preferred embodiments thereof, it is apparent to those skilled in the art that a variety of modifications and changes may be made without departing from the scope of the present disclosure which is intended to be defined by the appended claims.

What is claimed is:

1. A goggle structure, in which a side facing a wearer's face is defined as an inner side, and a side not facing the wearer's face is defined as an outer side, the goggle structure comprising:
a lens;
a frame provided with a pivot base on each side;
two adjustable linkage sets symmetrically pivoted on each side of the frame, wherein each of the adjustable linkage sets comprises a first arm body and a second arm body pivoted at one end to each other, another end of the first arm body is pivoted on the pivot base and is operable to pivot relative to the pivot base, and the second arm body is fixed to a side of the lens and is operable to pivot relative to the first arm body; and
two wearing bases symmetrically respectively provided on both sides of the frame for a wearing element to be assembled,
wherein the lens is operable to form spacing from the frame by means of the pivoting of the first arm body and the second arm body.

2. The goggle structure of claim 1, wherein when the first arm body and the second arm body are pivoted to a preset angle, the lens is lifted in a direction of an upper edge of the frame.

3. The goggle structure of claim 1, wherein the pivot base is provided with a plurality of first adjusting holes which are connected to each other, a first neck is formed between two adjacent first adjusting holes, the first adjusting holes collectively define a first arcuate track, the first arm body is provided with a first positioning portion, and the first positioning portion is selectively positioned within one of the first adjusting holes.

4. The goggle structure of claim 1, wherein the first arm body is provided with a plurality of second adjusting holes which are connected to each other, a second neck is formed between two adjacent second adjusting holes, the second adjusting holes collectively define a second arcuate track, the second arm body has a second positioning portion, and the second positioning portion is selectively positioned within one of the second adjusting holes.

5. The goggle structure of claim 1, wherein the frame is provided with at least one first magnet base, the first magnet base is provided with a first magnet, the second arm body is provided with a second magnet base, the second magnetic base is provided with a second magnetic, and when the second magnetic is attracted to the first magnetic, the lens is attached to an outside of the frame.

6. The goggle structure of claim 1, wherein the pivot base has a first pivot hole, an end of the first arm body is provided with a first hook, an end portion of the first hook is provided with a first protrusion annularly outwardly, the end portion of the first hook is axially provided with at least one first slot, the first hook is forced into the first pivot hole with the end portion thereof and is hooked to an inner side of the pivot base by the first protrusion, another end of the first arm body is provided with a second pivot hole, an end of the second arm body is provided with a second hook, an end portion of the second hook is provided with a second protrusion annularly outwardly, the end portion of the second hook is axially provided with at least one second slot, and the second hook is forced into the second pivot hole with the end portion thereof and is hooked to an inner side of the first arm body by the second protrusion.

7. The goggle structure of claim 1, wherein an upper edge and a lower edge of two sides of the lens are provided with opposing hole bases, respectively, each of the hole bases is provided with a fixing hole, an upper edge and a lower edge of the second arm body are provided with inserting members, respectively, and each of the inserting members is inserted into and fixed in the corresponding fixing hole.

8. The goggle structure of claim 7, wherein the fixing hole is polygonal in shape, and a profile of each inserting member matches a shape of the fixing hole.

9. The goggle structure of claim 1, wherein a top and a bottom of both sides of the frame are provided with snap holes, respectively, snap hooks extend from a top and a bottom of the wearing base, respectively, and the snap hooks are secured by hooking into the corresponding snap holes, respectively.

10. The goggle structure of claim 1, wherein the wearing base covers an outer side of the adjustable linkage set.
